(19) United States
(12) Patent Application Publication
(10) Pub. No.: US 2010/0312321 A1
(43) Pub. Date: Dec. 9, 2010

(54) Embolization coil and delivery system

(75) Inventors: Hiro Kiyosue, Yufu City (JP); Troels Qvist, Borup (DK)

Correspondence Address: COOK GROUP PATENT OFFICE, P.O. BOX 2269, BLOOMINGTON, IN, 47402, US

(73) Assignees: William Cook Europe ApS, Bjaeverskov (DK); Cook Incorporated, Bloomington, IN (US)

(21) Appl. No.: 12/455,548

(22) Filed: Jun. 3, 2009

(51) Int. Cl. A61F 2/06

(52) U.S. Cl. 623/1.11; 623/1.15

(57) ABSTRACT

A detachable embolization coil is screw threaded at the proximal end for releasable attachment to a delivery member within a delivery catheter. The coil has a proximal fibred portion and a distal non-fibred portion, the axial length of the non-fibred portion being as great or greater than the axial length of the non-fibred portion. The fibred portion carries a plurality of fibres of thrombogenic fibrous material. In this way, a partially delivered coil may be retracted into a delivery catheter for repositioning, substantially without hindrance from clotting.

### TECHNICAL FIELD

This application relates to coils for use in embolization procedures for treatment of a variety of conditions, including aneurysms.

### BACKGROUND OF THE INVENTION

Aneurysms present a potentially life-threatening problem. An aneurysm is the result of a weak area in a vessel wall, resulting in bulging in the weak area at a particular site in the vessel wall. Untreated aneurysms stand the risk of rupturing, which can result in a stroke or even death.

Endovascular embolization provides an alternative, non-surgical treatment for aneurysms or for other medical situations were vascular occlusion is desired. Typically, soft platinum coils (or stainless steel coils having increased radial strength) are deposited through a microcatheter into the aneurysm. The softness of the platinum minimizes ruptures and allows the coil to conform to the often irregular shape of the aneurysm or desired occlusion site. Generally, a number of separate coils are used to pack an aneurysm. The goal of this treatment is prevent blood flow into the aneurysm sac by filling the aneurysm with coils. Preventing blood flow eliminates the risk of rupture. By reducing blood flow, hemostasis and the formation of clots/thrombi can occur within e.g., the aneurysm or proximal to the aneurysm neck.

However, standard platinum embolization coils are biologically inert and are limited in their ability to promote thrombogenicity, or clotting. For this reason, there has been proposed the use with the embolization coil of fibre bundles of thrombogenic fibrous material.

The correct delivery of embolization coils can be difficult even after a catheter has been successfully steered to the region of the aneurysm or other intended site of vascoocclusion. The coil is necessarily flexible and steering of the distal end of the coil—for example into the neck of an aneurysm—can be troublesome. It will on occasion be desirable to retract the coil into the delivery catheter and reposition the distal tip of the coil more simply by repositioning the delivery catheter.

Because of the time necessarily taken to correctly steer the distal end of each separate coil, there will often be advantage in providing a given mass of embolization coil with a smaller number of longer coils rather than a larger number of shorter coils.

### SUMMARY OF THE INVENTION

In accordance with one aspect of the present invention, there is provided a detachable embolization coil comprising an elongate main coil body having a proximal end and a distal end, the proximal end of the main coil body being adapted for attachment to a delivery member such that the main coil body may be moved proximally and distally on, respectively, proximal and distal movement of that delivery member; the main coil body comprising contiguous fibred and non-fibred portions, the fibred portion extending axially from said proximal end and the non-fibred portion extending axially from said distal end, the axial length of the non-fibred portion being as great or greater than the axial length of the non-fibred portion, the fibred portion carrying a plurality of fibres of thrombogenic fibrous material, the non-fibred portion being clear of all thrombogenic fibrous material.

The fibred portion may have a length which represents from 15% to 50% of the total length of the coil. The fibres of thrombogenic fibrous material may have a length which is from 1.5 to 20 times the diameter of the coil. The coil may have a length greater than 5 cm; 10 cm; or 30 cm.

In another aspect of the invention, there is provided an embolization coil delivery system comprising a delivery catheter having a lumen opening to a distal end of the catheter; a delivery member mounted for proximal and distal movement longitudinally of the delivery catheter; a detachable embolization coil comprising an elongate main coil body having a proximal end and a distal end, the proximal end of the main coil body being releasably attachable to the delivery member; the main coil body comprising contiguous fibred and non-fibred portions, the fibred portion extending axially from said proximal end and the non-fibred portion extending axially from said distal end, the axial length of the non-fibred portion being as great or greater than the axial length of the non-fibred portion, the fibred portion carrying a plurality of fibres of thrombogenic fibrous material, the non-fibred portion being clear of all thrombogenic fibrous material.

In one embodiment, there is provided a detachable embolization coil is screw threaded at the proximal end for releasable attachment to a delivery member within a delivery catheter. The coil has a proximal fibred portion and a distal non-fibred portion, the axial length of the non-fibred portion being as great or greater than the axial length of the non-fibred portion. The fibred portion carries a plurality of fibres of thrombogenic fibrous material. In this way, a partially delivered coil may be retracted into a delivery catheter for repositioning, substantially without hindrance from clotting.

### BRIEF DESCRIPTION OF THE DRAWINGS

Embodiments of the invention will now be described by way of example with reference to the accompanying drawings, in which:

FIG. 1 is a somewhat diagrammatic view of an embolization coil according to one embodiment of the present invention, in a straightened configuration;

FIG. 2 is a view similar to FIG. 1, illustrating a J configuration of the coil;

FIG. 3 shows the coil of FIGS. 1 and 2 within a delivery catheter; and

FIG. 4 is a view similar to FIG. 3, depicting the detachment of the coil.

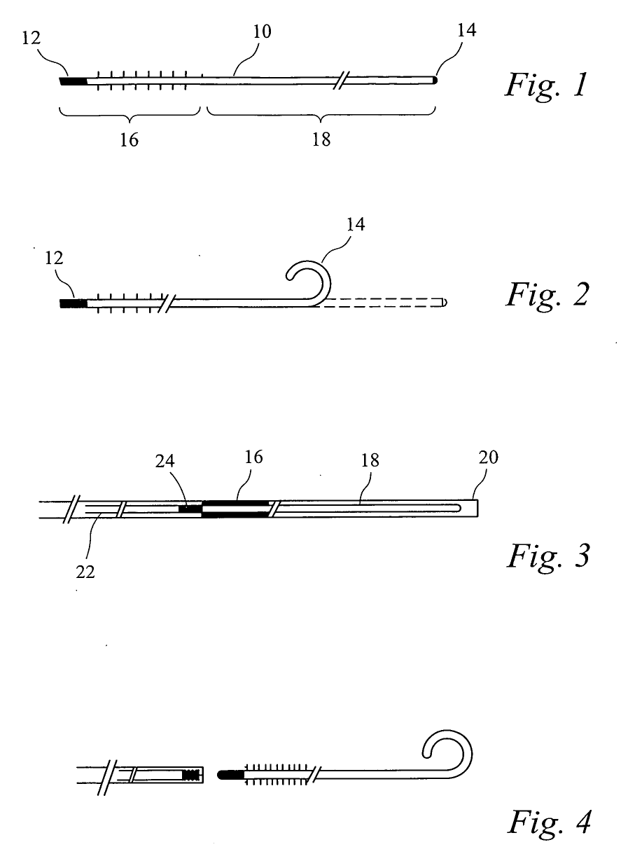

### DETAILED DESCRIPTION

Embolization coil 10 is formed in one example from coiled platinum or platinum alloy wire. It will be understood that the dimensions and parameters are variable to suit the intended usage of the embolization coil. In other examples, coils may be formed of other metals or metal alloy materials, containing stainless steel, gold; nickel-based alloys, such as NITINOL and INCONEL or other shape memory materials known in the art.

At the proximal end 12, the embolization coil is adapted by the formation of a screw thread to engage the distal end of a delivery member. Conveniently, the screw thread may comprise a male or female screw thread formed naturally at the outer or inner diameter of the coil. The total length of the coil may be 5 cm or more; 10 cm or more; 30 cm or more; as examples. The distal end 14 of the coil 10 may be formed with an atraumatic tip, by appropriate shaping of the end of the coiled wire, by addition of a rounded tip piece or by other appropriate means.

The coil has a fibred portion 16 extending from the proximal end 12 of the coil. In this fibred portion 16, the coil carries fibres of thrombogenic fibrous material. The term “thrombogenic fibrous material” here refers to a synthetic and/or natural fibrous material having thrombogenic properties. Exemplary thrombogenic fibrous materials include, but are not limited to, DACRON (Trade Mark), cotton, silk, wool, polyester thread and the like. The fibres are preferably carried as fibres or bundles of fibres which pass between adjacent turns of the coil, extending from the coil in two diametrically opposed directions. Conveniently, the fibres or fibre bundles are loaded onto the coil by the steps of: extending the coil longitudinally to separate each turn from its neighbouring turns; passing fibres or fibre bundles between adjacent turns to extend equal amounts on opposite sides of the coil; and allowing the coil to retract longitudinally to trap the fibres.

In certain examples, the length of fibres may be from 1.5 to 20 times the diameter of the coil. Preferably, there are from 50 to 200 turns between one fibre or fibre bundle and the next fibre or fibre bundle, along the length of the coil.

Importantly, there is a non-fibred portion 18 of the coil which is clear of thrombogenic fibrous material. This non-fibred portion 18 is contiguous with the fibred portion 16 and extends to the distal end 14 of the coil.

The fibred portion 16 has a length which is equal to or less than the length of the non-fibred portion 18. The fibred portion 16 may have a length which represents from 15% to 50% of the total length of the coil 10.

The coil may be heat-set or otherwise pre-shaped to assume a desired shape after at least initial delivery from the delivery catheter, to assist with steering of the distal end of the coil and/or to promote occlusion of a vessel. In one example, as depicted in FIG. 2, the distal region of the coil is pre-shaped to adopt a J configuration.

Referring now to FIG. 3, the coil 10 is positioned for delivery within the lumen of a delivery catheter shown schematically at 20. The proximal end of the coil 10 is releasably attached to the distal end of a delivery member 22, which extends generally coaxially within the delivery catheter 20. The distal end of the delivery member 22 is formed at 24 with a male or female screw thread to mate with the female or male screw thread at the proximal end of the coil 10.

In use, the delivery catheter is advanced to the intended site of vascoocclusion. This may be achieved in a variety of well known ways, for example by making use of a wire guide introduced percutaneously by the Seldinger technique. The delivery catheter 20 is then retracted proximally with respect to the delivery member 22 to deliver from the catheter the distal end 14 of the coil and a distal region adjacent the distal end. The distal end 14 of the coil is then maneuvered into position, through for example the neck of an aneurysm which it is desired to occlude. The described pre-shaping at the distal end of the coil may assist in this manoeuvre.

Sometimes, the attempt to manoeuvre the distal end of the coil will prove unsuccessful. Other times the initial attempt to manoeuvre will be successful but the coil becomes misplaced as the length of the coil begins to be delivered. In these instances, the physician will wish to retract the coil into the delivery catheter and restart the procedure. Because the region of the coil adjacent the distal end is non-fibred, there is a minimal risk of such attempt to retract the coil being prevented or complicated by clotting. It will be understood that any thrombogenic fibrous material present on that part of the coil that has been delivered from the delivery catheter would promote clotting. Any significant clotting would lead to an increased frictional force on the coil and on the thrombogenic fibrous material, acting to resist the attempted retraction. Retraction would be more difficult and sometimes not possible. Retraction after significant clotting in the distal region of the coil may also result in damage to the coil through over-extension.

It will thus be recognised that the described arrangement provides a coil which will promote significant thrombogenicity once completely delivered, yet which should allow the physician several attempts at delivery. The described arrangement therefore provides a solution for the delivery of long, highly flexible coils—including liquid coils—for a variety of vascular occlusion and other embolization procedures. The coils may be shaped in J or other configurations, or unshaped.

A screw thread attachment between the proximal end of the coil and the delivery member is convenient, particularly if use is made of the natural screw thread formation of the coil. Of course, the coil body may be adapted in other ways for attachment to a delivery member such that the main coil body may be moved proximally and distally on, respectively, proximal and distal movement of that delivery member.

What is claimed is:

1. A detachable embolization coil comprising an elongate main coil body having a proximal end and a distal end, the proximal end of the main coil body being adapted for attachment to a delivery member such that the main coil body may be moved proximally and distally on, respectively, proximal and distal movement of that delivery member; the main coil body comprising contiguous fibred and non-fibred portions, the fibred portion extending axially from said proximal end and the non-fibred portion extending axially from said distal end, the axial length of the non-fibred portion being as great or greater than the axial length of the non-fibred portion, the fibred portion carrying a plurality of fibres of thrombogenic fibrous material, the non-fibred portion being clear of all thrombogenic fibrous material.

2. The coil of claim 1, wherein the fibred portion has a length which represents from 15% to 50% of the total length of the coil.

3. The coil of claim 1 or claim 2, wherein the fibres of thrombogenic fibrous material have a length which is from 1.5 to 20 times the diameter of the coil.

4. The coil of any one of claims 1 to 3, having a length greater than 5 cm; 10 cm; or 30 cm.

5. An embolization coil delivery system comprising a delivery catheter having a lumen opening to a distal end of the catheter; a delivery member mounted for proximal and distal movement longitudinally of the delivery catheter; a detachable embolization coil comprising an elongate main coil body having a proximal end and a distal end, the proximal end of the main coil body being releasably attachable to the delivery member; the main coil body comprising contiguous fibred and non-fibred portions, the fibred portion extending axially from said proximal end and the non-fibred portion extending axially from said distal end, the axial length of the non-fibred portion being as great or greater than the axial length of the non-fibred portion, the fibred portion carrying a plurality of fibres of thrombogenic fibrous material, the non-fibred portion being clear of all thrombogenic fibrous material.

6. The coil delivery system of claim 5, wherein the fibred portion has a length which represents from 15% to 50% of the total length of the coil.

7. The coil delivery system of claim 5 or claim 6, wherein the fibres of thrombogenic fibrous material have a length which is from 1.5 to 20 times the diameter of the coil.

8. The coil delivery system of any one of claims 5 to 7, having a coil length greater than 5 cm; 10 cm; or 30 cm.

9. A method of embolization coil delivery system utilising a delivery catheter having a lumen opening to a distal end of the catheter; a delivery member mounted for proximal and distal movement longitudinally of the delivery catheter; and a detachable embolization coil comprising an elongate main coil body having a proximal end and a distal end, the proximal end of the main coil body being releasably attachable to the delivery member; the main coil body comprising contiguous fibred and non-fibred portions, the fibred portion extending axially from said proximal end and the non-fibred portion extending axially from said distal end, the fibred portion carrying a plurality of fibres of thrombogenic fibrous material, the non-fibred portion being clear of all thrombogenic fibrous material, the method comprising the steps of manipulating the catheter and delivery member to partially deliver the coil as many times as is necessary to achieve correct placement, with the fibred portion remaining within the delivery catheter during the or each partial delivery; withdrawing the entirety of the coil into the catheter where necessary for repositioning; and on correct placement fully delivering the coil with detachment from the delivery member.
